(19) United States
(12) Patent Application Publication
(10) Pub. No.: US 2004/0133137 A1
(43) Pub. Date: Jul. 8, 2004

(54) Non-compression carpal/wrist keyboard brace

(76) Inventors: Veronica Rose Hargis, Warwick, NY (US); Luther Calvin Hargis, Warwick, NY (US)

Correspondence Address: DR. LUTHER CALVIN HARGIS, 40 MAPLE AVE., WARWICK, NY, 10990, US

(21) Appl. No.: 10/724,549

(22) Filed: Nov. 28, 2003

Related U.S. Application Data
(60) Provisional application No. 60/430,535, filed on Dec. 4, 2002.

(51) Int. Cl. A61F 5/00

(52) U.S. Cl. 602/21

(57) ABSTRACT

A carpal tunnel device for supporting the wrist and hand utilizing a raised dome to prevent contact or compression to the carpal tunnel area. The device consisting of a clear thin thermoplastic shell, and an adjustment strap for attaching the device to the wearer. Contouring the thenar and hypothenar area of the palm, lateral wrist and proximal forearm a mid section of raised dome is formed over the carpal tunnel area of the wearer allowing great freedom of movement, ultra light weight, non-compression from elastomeric or circumferential glove forces, slim non bulky design, and complete tunnel non-compression from sharp of irregular surfaces or protrusions of the work environment. Thus the wearer may rest the inner wrist on counters, keyboard surfaces, counter edges or sharp and irregular surfaces without carpal area compression. Worn at night, the brace prevents compression of the carpal area as the wearer places his hand and wrist under his head or below his pillow.

This application claims the benefit of U.S. Disclosure Document 508091 and Provisional Patent 60/430,535

### FIELD OF THE INVENTION

The present invention relates to a method and apparatus for supporting a wrist, and more particularly to a wrist support used for abating the affects of sustained or intermittent pressure to the carpal tunnel from sharp, irregular or heavy objects that compress the carpal tunnel area and lead to carpal tunnel syndrome.

### BACKGROUND OF THE INVENTION

Carpal tunnel syndrome is a common condition of the hand caused by direct compression, bruising and swelling of the synovial membranes, tendons and nerves of the carpal tunnel region, and by any condition that reduces the available space in the carpal tunnel. Prolonged compression of the inner wrist from many consecutive hours of resting the wrist on hard or irregular surfaces in clerical, key boarding and computing duties can lead to over-compression of the carpal tunnel and its median and ulnar nerves.

Excessive flexion of the wrist and fingers, such as repetitive and forceful grasping of the hands and repetitive bending of the wrist, are common causes of carpal tunnel syndrome. Carpal tunnel syndrome can also be caused from injury or trauma such as blunt contusions, wrist bone dislocations and fractures which compromise the carpal tunnel, thereby resulting in pressure on the median nerve. Common symptoms of this condition include pain and numbness of the hand and fingers. Specifically, these symptoms are worsened by the confined nature of the carpal tunnel itself being bounded by carpal bones at its dorsal aspect and by a dense fibrous tissue called the transverse carpal ligament at its volar aspect leaving little room for the delicate carpal nerves.

Treatment of carpal tunnel syndrome varies according to the severity of the condition. Severe conditions usually require hand surgery to sever the transverse carpal ligament. For less severe cases, the use of a splint, which immobilizes the wrist, is sometimes effective, often in combination with anti-inflammatory medication. Such treatments are generally expensive, painful and may reduce the patient's ability to use the affected hand.

Carpal tunnel syndrome is a particular problem for workers in industries which require repeated manual hand motions and prolonged postures of resting the inner wrists to hard surfaces, such as modern day clerical and keyboard operations. In response to this problem, keyboard wrist rest and wrist devices have become ubiquitous.

The keyboard wrist rests are intended to keep the hand and wrist at about a 10 degree angle, which is referred to as the neutral position. However, the keyboard wrist rests provide little benefit and in fact may promote the problem as they allow the weight of the hand and forearm to be rested centrally atop the carpal tunnel area in many cases.

Wrist devices for the treatment or prevention of carpal tunnel syndrome include bracelets, gloves and wrist support. These devices have limitations such as, impeding free motion of the hand and digits of the hand, are often uncomfortable to wear, do not usually prevent the wrist from dropping below the neutral position, are often cumbersome and become snagged on sharp or hooked objects, are often hard to remove and re-apply, or heavy in weight.

Another very large problem with these devices is that they allow for the volar wrist and carpal tunnel region to come into contact with the inner aspect of the brace itself and thereby transfer pressure from the resting surface through the brace to the carpal tunnel space. If glove like in nature these braces, even those with raised ribs, allow for excessive load to the carpal tunnel region if the wrist is placed over an irregular or projecting surfaces

A further disturbing result or elastomeric gloves including those with raised ridges is that when the hand is placed below the head or pillow as in sleep positions the weight of the human head transmits through and into the glove compressing the carpal tunnel.

Bracelets and gloves create circumferential pressure points around the wrist and are often touted to alleviate carpal tunnel syndrome; however, circumferential pressure can be uncomfortable to the wearer and created added compression of the carpal tunnel region. This is especially of elastomeric gloves that generate circumferential forces that pressurize the carpal tunnel while worn.

Because elastomeric gloves are non-rigid they are easily compressible from external forces acting on them from any direction and can become displaced or skewered by these forces.

Traditional wrist supports enclose the wrist and at least a portion of hand. Since the hand is partially encased, these devices unnecessarily restrict hand mobility and are uncomfortable to wear.

Additionally, wrist supports typically utilize a rigid stay to maintain the wrist in the neutral position. The stay in conventional wrist supports generally is too narrow and causes pressure points and ridge-lined surfaces which are uncomfortable, or, the stay is too wide and will not bend or conform to the circumference of the wrist.

Another consideration is that the wrist device should provide for weight dispersal to the less sensitive portions of the hand and forearm while sparring compression to the delicate carpal tunnel region. Although braces are adapted to hold the carpal area, wrist, hand and forearm in a fixed or semi-fixed alignment they contact the carpal tunnel on the inner aspect of the brace allowing for the transmittal of arm pressure through the brace and to the delicate carpal tunnel region.

Accordingly, what is needed is a device for the treatment and prevention of carpal tunnel syndrome which keeps the wrist and hand in a relatively neutral position. Also a device that will not unduly impede movement and use of the hand and digits to facilitate ease of work and productivity. Additionally the brace must protect the carpal tunnel allowing no compression what so ever to the area from flat, sharp, irregular or heavy objects such as the head and thereby transfer force, weight and pressure to less sensitive portions or the hand where delicate nerves are not prone to compression. Further, the device should be comfortable to wear, light in weight, and unlikely to become caught or snagged on sharp or projecting objects.

### DESCRIPTION OF THE PRIOR ART

This applicant is aware of the following U.S. patents concerning wrist support devices.

U.S. Pat. No. Inventor Issue Date Title U.S. Pat. No. 5,769,804 Harris et al. Jun. 23, 1998 CARPAL TUNNEL SYNDROME WRIST BRACE: U.S. Pat. No. 5,746,707 Donald Eck May 5, 1998 CARPAL TUNNEL SYNDROME EXTERNAL BRACE: U.S. Pat. No. 5,468,220 Benjamin Sucher November 21, CARPAL TUNNEL 1995 BRACELET: U.S. Pat. No. 5,417,645 Roger Lemmen May 23, 1995 FLEXIBLE WRIST SPLINT FOR CARPAL TUNNEL SYNDROME TREATMENT: U.S. Pat. No. 6,517,501 Gerald Slautterback Feb. 11, 2003 CARPAL TUNNEL DEVICE AND METHOD: U.S. Pat. No. 5,810,753 Eberbach Sep. 22, 1998 GLOVE: U.S. Pat. No. 5,385,537 Mark Davini Jan. 31, 1995 SPLINT SYSTEM: U.S. Pat. No. 5,160,314 Helena Peters Nov. 3, 1992 WRIST SUPPORT

Harris et al., U.S. Pat. No. 5,769,804, discloses a carpal tunnel syndrome wrist brace to immobilize the wrist after carpal tunnel syndrome surgery. The brace includes a shell having first and second edges lined with a plurality of eyelets and both of the edges being connected together by a tongue stitched there between. The shell includes a thumb hole for receiving a thumb and is shaped to fit a patient's wrist such that the patient can insert the hand and wrist into the proximal end of the shell and extend four fingers through the distal end of the shell with the thumb through the thumb hole. The shell is then secured to the patient's wrist to activate immobilization of the wrist using a pair of hook and loop fasteners and a plurality of laces.

Eck, U.S. Pat. No. 5,746,707, teaches a wrist restraining device for the treatment of carpal tunnel syndrome wherein the device comprises a palmer brace which restrains the wrist, thumb and fifth digit, with unrestricted motion of the second, third, and fourth digits, to relieve pressure on the median nerve in the wrist.

Sucher, U.S. Pat. No. 5,468,220, provides a bracelet-like device for the treatment or prevention of carpal tunnel syndrome where the device includes a C-shaped member having a central portion located over the dorsal side of the wrist and two arms encircling the wrist. Each arm has a pad for engaging the palm near the attachment edges of the carpal ligament at the medial border and the lateral border of the carpal bones. The device also includes a pad attached to the central portion for engaging the dorsal part of the wrist.

Lemmen, U.S. Pat. No. 5,417,645, discloses a flexible splint for the treatment of carpal tunnel syndrome. The splint includes an elongate, flexible member having a palmer portion and an elongate proximal portion. The palmer portion has a curved sickle shape and defines a thumb notch. The palmer portion is angled with respect to the proximal portion to position the hand in a near normal anatomical position. Elastic straps attach the splint to the wrist and palm of the user.

Slautterback, U.S. Pat. No. 6,517,501, provides a carpal tunnel device for supporting the hand in a neutral position to alleviate and prevent the carpal tunnel syndrome. The device includes a pad for placement against a wearer, a base attached to the pad, a stay disposed between the pad and base, and an adjustment strap for attaching the device to the wearer. The palmar section of the brace having a width sufficient to prevent opposition of the thump and fifth finger of the wearer.

Eberbach, U.S. Pat. No. 5,810,753 relates an elastomeric glove with raised parallel elastomeric ridges over the lateral volar surface of the wrist to disperse forces away from the carpal region when the wrist is placed against flat, sharp but not irregular of heavy downward forces

Davini, U.S. Pat. No. 5,385,537, discloses a splint including a brace which cradles either the radius or the ulna of the wrist and an attachment strap which embraces the other two bones. The brace and the strap act to approximate the radius and the ulna toward one another to support the carpal tunnel without allowing compression of the anterior surface of the carpal tunnel.

Peters, U.S. Pat. No. 5,160,314, teaches a wrist support adapted to be pulled onto and positioned about the hand and wrist area and adapted to anatomically conform to the wrist and basal hand of the wearer. The wrist support includes a sleeve constructed of a resilient elasticized fabric. The sleeve is provided with a thumb opening and means for receiving a palmar stabilizing stay, with the stay being removable. A compression strap is integral with the sleeve and extends around the wrist.

### SUMMERY OF THE INVENTION

The present invention is a device for preventing and alleviating carpal tunnel syndrome. During normal operation, the invention is positioned across the wrist of the wearer to support the hand and wrist in the neutral position while restricting hand flexion slightly. While worn, the device allows the wearer free motion of the wrist in all other directions not conducive to carpal tunnel syndrome, full use of the hand and digits of the hand, and a degree of comfort and lightness of weight not provided for in prior devices. The carpal tunnel device also allows for a raised dome above the carpal tunnel area to prevent any pressure to the area and to allow for the displacement of weight away from that area to the palm, lateral wrist and proximal forearm. Thus the wearer may rest his or her hand and forearm upon hard, sharp or irregular or pointed surfaces and projections for prolonged periods without compression of the carpal tunnel region. Additionally the wearer cannot compress the carpal tunnel even in positions of extreme wrist extension. As desired the brace is extremely easy to remove or re-attach. Further the wearer may adopt sleep position wherein the head is placed atop the hand and wrist without transmitting the weight of the head to the carpal tunnel to compress and injure the area.

In the broadest sense, the carpal tunnel device includes a palmar section for extending over a heel of a hand and mid way forward of the thenar and hypothenar eminences, and a main section which extends from the palmar section for engaging the forearm of a wearer. More preferably, the palmar section of the carpal tunnel device has a contour that fits or cups the hypothenar eminence and a thenar eminence. The mid section is domed in conformation and therefore allows no compression from external surfaces. Equally important the inner aspect of the brace is not in contact with the carpal area and thus forces acting on the external brace are not allowed to be transmitted through the shell of the brace to affect the carpal area.

### OBJECT OF THE INVENTION

The principal object of the present embodiment is to provide a device for the prevention and alleviation of carpal tunnel syndrome.

Another object of this invention is to provide an apparatus for supporting the wrist and hand in a neutral position.

A further object of this device is to provide a brace which does not markedly impede movement of the hand and digits of the hand which is not associated with carpal tunnel syndrome.

Another object of this invention is to provide a support which allows no compression to the carpal area from external sources or the inner wrist itself even when the wrist is extended or dorsi flexed.

Another object of this invention is to provide a device which allows no contact of the inner brace components to the carpal tunnel area that would transmit the pressure of external surfaces through the brace.

A further object of this invention is to provide a device which is comfortable to wear.

Another object of the brace is to provide a device that is extremely light in weight.

A final object of the brace is to provide a support that is curvilinear and not subject to becoming caught or snagged by rough, sharp or pointed projections.

### BRIEF DESCRIPTION OF THE DRAWINGS

FIG. 1 is a frontal view of the invented non-compression carpal/wrist keyboard brace in operating position attached to a wearer.

FIG. 2 is a perspective view of the device of FIG. 1 taken from the inside of the wearer's wrist.

FIG. 3 is a perspective of the carpal tunnel device from a looking at the inside of the brace that would contact the wearer.

FIG. 4 is a frontal and slightly downward view of the carpal tunnel device.

FIG. 5 is a view of the typical molding chamber, positive mold and thermoplastic sheet.

### DETAILED DESCRIPTION

The present invention is a device for preventing and alleviating carpal tunnel syndrome. In particular, the present invention attaches at the wrist of a wearer to support the wrist and hand in the neutral position. While worn, the device allows the wearer free motion of the wrist in directions unrelated to carpal tunnel syndrome, full use of the hand and digits of the hand, and a greater degree of comfort than provided in prior devices.

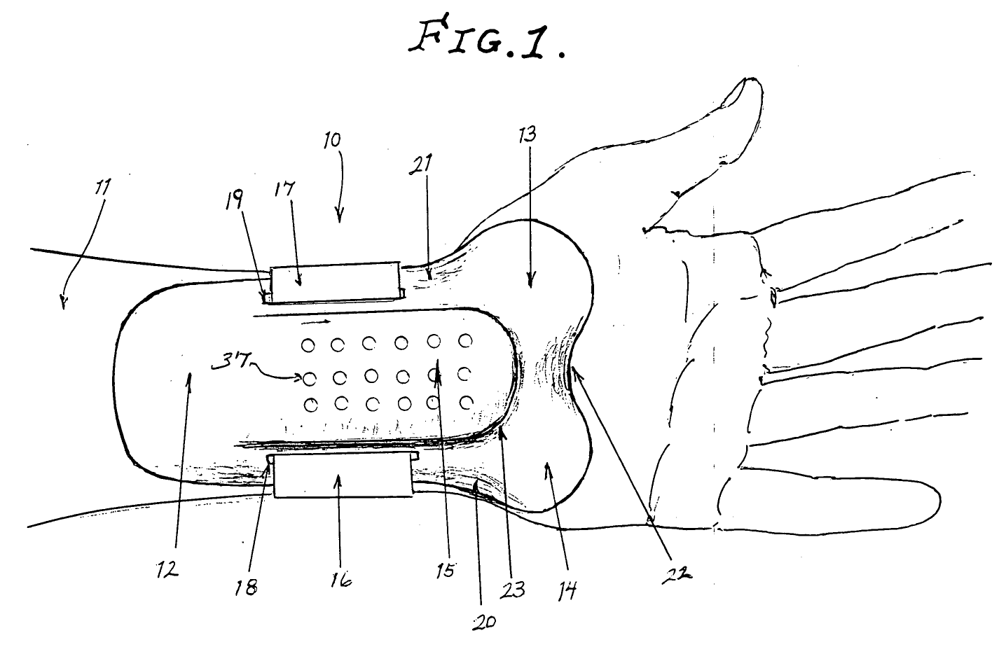

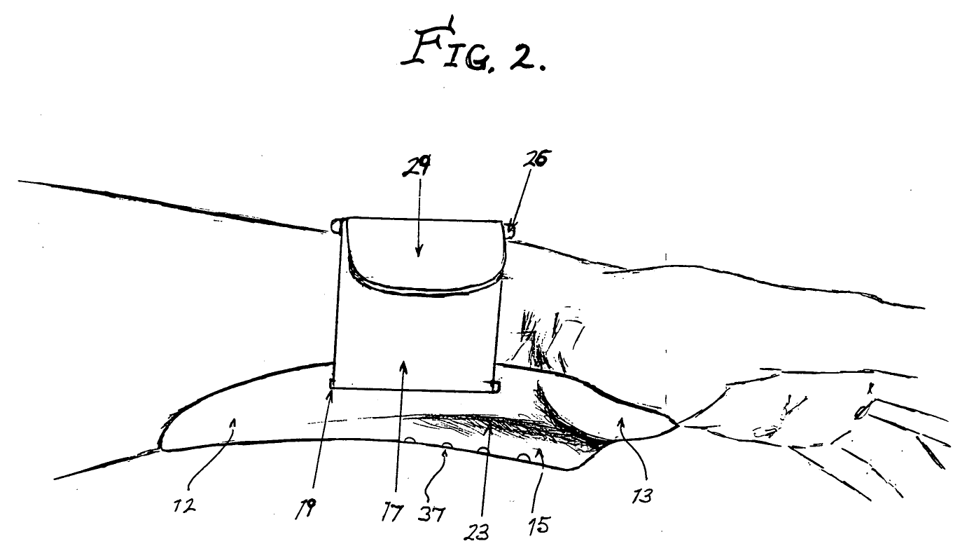

Referring now to the drawings, and particularly FIGS. 1 and 2, the invented universal carpal tunnel device 10 includes a palmar section 13 and 14 for engaging the heel of a wearer's hand at the thenar and hypothenar eminences respectively, a main section comprising areas 15, 20 and 21 extending rearward from the palmar section for engaging the forearm of the wearer, a rearward section designated 12 for resting against the forearm 11, and a means for removably attaching straps 16 and 17 of the carpal tunnel device 10 to the wearer.

The main section 21 and 20 have a narrowed and contoured area at the wrist-hand junction of the wearer for contacting the lateral wrist just outside of the carpal tunnel area and extend partially along the forearm of the wearer, and a raised or domed area indicated as 15 that acts as a shield to prevent compression on the carpal tunnel region. The width of the main section is sufficient to comfortably cradle the lateral forearm of the wearer. The main section has additional features; ventilation holes designated 37, slots 18 and 19 for passage of the wrist straps 16 and 17, and a contoured margin 23 at the border of the protect raised dome 15 which demarcates the area where the sides of the raised dome elevate away dome the lateral margins of the brace midsection 20 and 21.

The palmar section 13 tapers forward from the distal end of the main section and extends at least partially over the heel of the hand at the thenar eminence where it contours the shape of this anatomic area. The palmar section 14 tapers forward from the distal end of the main section and extends at least partially over the heel of the hand at the hypothenar eminence where it contours the shape of this anatomic area. These two areas are connected by an area assigned 22 that contours the mid area or the palm between the thenar and hypothenar regions. The palmar sections have sufficient width to provide support for the center of the thenar and hypothenar eminences thereby displacing the weight of the resting arm and hand away from the carpal tunnel and to the heel of the palm.

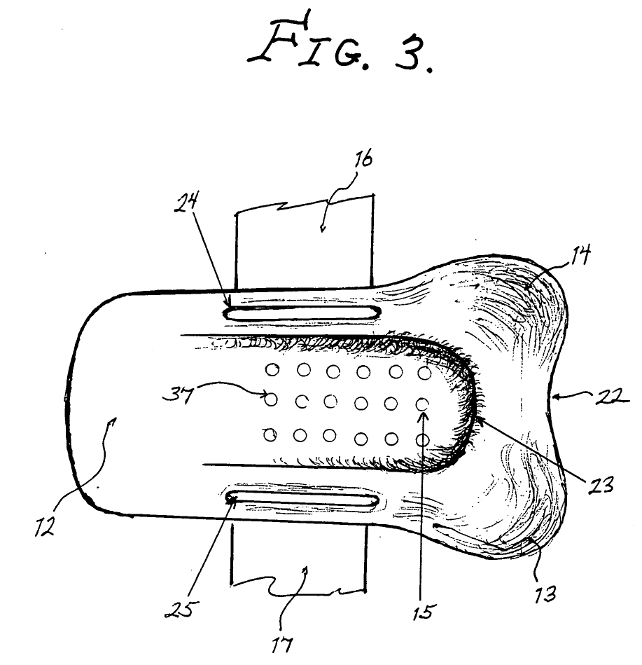

FIG. 3 is and inside view of the carpal tunnel device 10. This view shows the general contour of the contacting area of the brace to the wearer. 23 indicates the demarcation of the rim of the domed area of the brace 15 which would form a cavity in this perspective. Also indicated are areas 24 and 25 which are the terminal areas of the straps 16 and 17 where the straps pass through the slots 18 and 19. The terminal ends of the straps 24 and 25 are made large enough to prevent their passing through the slots 18 and 19. It should be understood that straps 16 and 17 would extend to be fastened atop the wrist and forearm by and convention means.

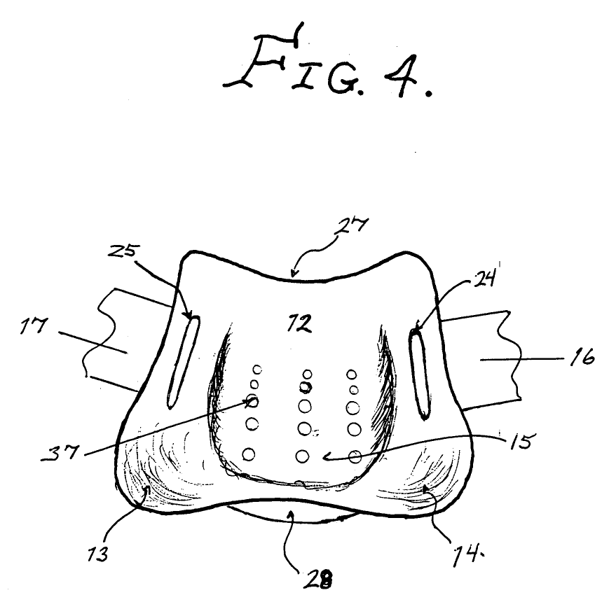

As shown in FIG. 4, the carpal tunnel device from the front and the inner perspective. The thenar and hypothenar areas 13 and 14 are seen to be curved as previously described to contour the eminences they are named for. The area designated 28 is the downward curved outer surface of the dome 15. The rearward edge of the device is assigned 27.

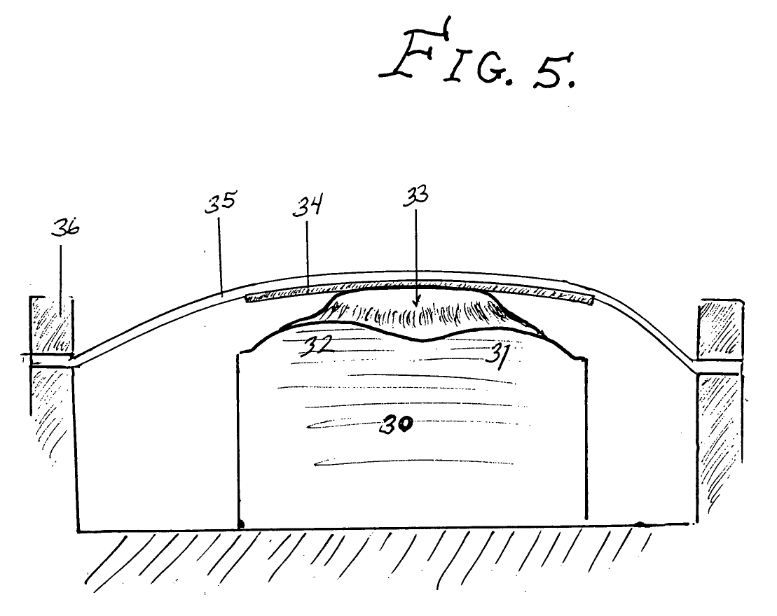

FIG. 5 indicates the standard vacuum molding process where; 36 is the wall of the vacuum mold, 35 is the elastic membrane of the mold which contours the heated plastic 34 to the positive mold 30. The positive mold has contours 32 and 31 reflecting the thenar and hypothenar surfaces respectively and an area 33 corresponding to the raised dome 15 of the device.

### SUMMERY OF THE ACHIEVEMENT OF THE OBJECTS OF THE INVENTION

From the foregoing, it is readily apparent that I have invented a non compression carpal tunnel wrist support device for alleviating and preventing carpal tunnel syndrome. The non compression carpal/wrist keyboard support device can be worn to maintain the hand at the neutral position, support the hand at the proper typing height, to not unnecessarily restrict movement of the hand and digits of the hand which are unrelated to carpal tunnel syndrome, and to prevent compression either externally from work surfaces or internally from brace components to the delicate area of the carpal tunnel as a result of external or internal forces acting upon the wrist and hand.

It is to be understood that the foregoing description and specific embodiments are merely illustrative of the best mode of the invention and the principles thereof, and that various modifications and additions may be made to the apparatus by those skilled in the art, without departing from the spirit and scope of this invention.

What is claimed:

1. A thermoplastic shell splint or brace for the carpal tunnel, comprising:
a) a support and protective shield/dome overlying the carpal tunnel of the human wrist
b) first securing means for reasonably securing the support means to the hand.
c) a thin shell formed of a rigid material fitted to the human hand and forearm to rest upon the thenar, hypothenar, lateral distal wrist and proximal forearm
d) a dome or raised shield protecting the carpal tunnel arising from the portions of the brace in contact with the skin of statement c above

2. The splint of claim 1, wherein the support means is rectangular and spanning the proximal palm, volar surface of the wrist and the proximal forearm

3. The splint of claim 2, wherein the support means include two straps secured to the lateral margins of the brace and joined to the dorsal area of the wrist by a buckle/velcro closure to secure the brace

4. The splint of claim 2, having longitudinal and transverse axes for the treatment of carpal tunnel syndrome, comprising: a palmar section having a distal end contoured to fit the rounded surfaces of the thenar and hypothenar areas of the palm and to end forward approximately mid way of these anatomic landmarks; a mid section extending from the proximal end of said palmar section forming a raised dome over the area of the mid inner wrist or carpal tunnel region of the wearer which slopes downward at its lateral edges to contact the outer portion of the wrist; a proximal section extending backwards from the said mid section to contour the forearm

5. The carpal tunnel device according to claim 1 wherein said dome is perforated by small holes for ventilation of the wearer's skin.

6. The carpal tunnel device according to daim 1 wherein the mid section of the brace is a raised dome above the carpal area elevated above and away from the width and breath of the of carpal tunnel area approximate five millimeters at the domes forward aspect and tapering backwards gradually to conform with and to lie against the forearm at the rearward or proximal border of the carpal tunnel area at the forearm.

7. The carpal tunnel device according to claim 6 wherein said dome curves downwards and outwards to contact the proximal base of the hand just forward of the mid wrist crease, laterally the outer borders of the volar wrist just outside the lateral borders of the anatomic carpal tunnel, and rearward the forearm just proximal to the anatomic carpal tunnel area.

8. The carpal tunnel device according to claim 3 wherein said attachment means is a single strap for wrapping about the forearm having a first and second end secured to said main section and ascending from either side of the brace to fasten by the passing of the second strap through a buckle ending of the first strap and then pulling of the second strap to fasten upon itself by means of Velcro closure.

9. The carpal tunnel device according to claim 1 wherein the shell or base is composed of a thin low temperature molded thermoplastic having a gage of approximately 1/16 inch depending on thickness desired.

10. The carpal tunnel device according to claim 2 wherein the shell is contoured to follow the natural conformation of the palm, wrist and hand, except at the domed portion of the mid section which is raised above the tunnel; and is generally linear along the longitudinal and transverse axes and is curvilinear in a direction normal to the axes to support the hand in a neutral position.

11. The carpal tunnel device according to claim 7 wherein said carpal tunnel device is self-supporting in shape but ultra light in weight at 1.5 to 2 ounces and rigid in character so as to maintain its shape and support characteristics under load bearing.

12. The carpal tunnel device according to claim 1 that is contoured to a minimal area of the palm, wrist and forearm to allow for a greater range of motion while protecting the carpal region.
